(19) United States
(12) Patent Application Publication
(10) Pub. No.: US 2026/0215772 A1
(43) Pub. Date: Jul. 30, 2026

(54) MINIMALLY-INVASIVE SUTURING DEVICE

(71) Applicant: KOVE Medical AG, Zürich (CH)

(72) Inventors: Yannick Devaud, Zürich (CH); Helena Tronnier, Zürich (CH); Daniel Cosovanu, Marginea (RO); Alexandre Ioan Romoscanu, Genève (CH); Ryan Vincent Tiensing Young, Dresden (DE); Kurt Ruffieux, Thalwil (CH)

(21) Appl. No.: 19/142,396

(22) PCT Filed: Dec. 21, 2023
(86) PCT No.: PCT/EP2023/087358

§ 371 Date: Jun. 23, 2025

(30) Foreign Application Priority Data
Dec. 22, 2022 (EP) 22216172.1

(51) Int. Cl. A61B 17/04; A61B 17/00; A61M 25/00

(52) U.S. Cl. CPC A61B 17/0482; A61B 2017/00358; A61B 2017/00623; A61B 2017/00663; A61B 2017/0472; A61M 25/0068; A61M 2025/0095

(57) ABSTRACT

The invention relates to a device to be used for closing a tissue opening in a tissue. In particular, the present invention relates to a device used for a closing an opening in a membrane by stitching in a minimally invasive procedure.

### CROSS-REFERENCE TO RELATED APPLICATION(S)

The present application is a § 371 national phase entry of International patent application Serial No. PCT/EP2023/087358, filed Dec. 21, 2023, and published in English, and further claims priority to European patent application 22216172.1, filed Dec. 22, 2022.

### BACKGROUND OF THE INVENTION

The invention relates to minimally-invasive suturing device for closing a tissue opening in a tissue. In particular, the present invention relates to a minimally-invasive suturing device for closing an opening in a membrane by stitching.

The minimally-invasive closing of tissue openings remains a challenge in various medical procedures.

For example, one such procedure is the suturing of a fetal membrane in fetoscopy and/or fetal surgery. The fetal membrane is a relatively thin tissue layer having a thickness of a few hundred microns that surrounds the fetus during development. There is a need to prevent iatrogenic preterm premature rupture of the fetal membrane. In open fetal surgery, the fetal membrane is attached to the uterus along the surgical opening to avoid large scale detachment from the uterus. Following the surgery on the fetus, the uterus is closed using sutures, typically resorbable sutures. Minimally-invasive fetal interventions are believed to be beneficial. For example, it is believed that the limited size of the incision may significantly reduce the wound healing time, pain and remaining scar. However, there continues to be a need for suitable procedures to attach the fetal membrane to the uterus and close the tissue defect created by an access puncture in such minimally-invasive approaches, e.g., where the surgery is performed through a sheath introducer.

Devices for stitching various other tissue openings in minimally invasive procedures are known. Prior art stitching devices are known, for example, from WO 2012/142338 A2, which relates to suturing devices for suturing an anatomic valve. EP 2 412 317 A1 relates to a wound closure device for suturing an opening in tissue. U.S. Pat. No. 8,992,549 B2 relates to intra-abdominal suturing devices designed for closing puncture wounds created by surgical trocars and similar puncturing devices. U.S. Pat. No. 10,413,296 B2 relates to devices, systems, and methods for closing a wound or opening in tissue.

Each of these devices, however, is believed to have certain disadvantages in use. Moreover, none of these devices is believed to be well-adapted for suturing a tissue opening in minimally invasive fetal surgeries. Improved devices for closing an access opening in fetal interventions would thus be desirable. Moreover, improved devices for closing openings in other tissues and/or in minimally-invasive procedures in other regions of the body would be desirable.

Disclosed embodiments of the present invention provide an improved device and method for closing a tissue opening in a tissue with at least one suture.

Disclosed embodiments of the present invention provide an improved device and method for closing an opening created for minimally invasive fetal procedures.

Disclosed embodiments of the present invention provide a device and method that is easily and safely operable, for example easily and safely operable within the uterus.

Disclosed embodiments of the present invention provide a device and method that can be operated by a single user in an intuitive and consistent manner.

Disclosed embodiments of the present invention provide a device that reduces the complexity of a suturing procedure for the user and provides for a fail-safe operation.

Disclosed embodiments of the present invention provide a device and method allowing for suturing without requiring additional supporting equipment, for example in fetal procedures, such as one or more cameras for visualization (e.g., fetal endoscopy cameras). It is a further object of the present invention to thereby reduce the overall costs of a respective medical procedure.

At least some of these benefits are achieved by the devices and methods according to the disclosed embodiments of the invention described herein.

### SUMMARY OF THE INVENTION

According to a first aspect of the present invention, a device for closing a tissue opening in a tissue is disclosed. The tissue extends at least partially around a body cavity or lumen.

The device comprises an elongate shaft having a proximal end and a distal end. The elongate shaft extends along a longitudinal axis and is configured to be inserted into the tissue opening in the tissue. The device further comprises at least one first and at least one second needle guide, each needle guide having an exit opening at its distal end, at least one first hollow needle and at least one second hollow needle, each of the first and second needles being configured to be translated along and advanced out of a respective one of the needle guides in a distal and radially outward direction from a retracted position to an extended position, at least one first snare configured to be advanced out of a distal opening of the at least one first hollow needle, and at. least one second snare configured to be advanced out of a distal opening of the at least one second hollow needle.

Optionally, the needles are configured to penetrate the tissue from outside the body cavity or lumen towards the inside. It is believed that this may allow to provide a simple and safe actuation mechanism. A handle of the device may comprise one or more actuators for moving the needles in the distal and/or proximal direction.

The at least one first snare and/or the at least one second snare may be configured and/or pre-shaped to extend in a radially inward direction towards the elongate shaft when advanced out of a distal opening of the at least one first hollow needle. Alternatively or additionally, the device may be configured such that a distal end of the first and/or second snare is disposed distally of the distal end of the elongate shaft when the respective snare is in a fully advanced position. Such configuration of the snares may be helpful in providing a reliable capturing of a suture, as explained in more detail hereinbelow. The handle may comprise one or more actuators for moving the first and/or second snares in the distal and/or proximal direction.

The device according to the first aspect of the present invention may further comprise at least one first snare sleeve configured to be advanced out of a distal opening of the first needle and at least one second snare sleeve configured to be advanced out of a distal opening of the second needle. Optionally, the at least one first snare is configured to be advanced out of a distal opening of the first snare sleeve, and the at least one second snare is configured to be advanced out of a distal opening of the second snare sleeve. The first snare sleeve may be configured to be advanced along and/or close the first snare, and the second snare sleeve may be configured to be advanced along and/or close the second snare, for example while the first and second snares, respectively, are kept in position. Such arrangement may be helpful for closing the respective snare once the suture has been captured therein and/or avoiding the suture from being pulled out of the snares. The handle may comprise one or more actuators for moving the first and/or second snare sleeves in the distal and/or proximal direction.

The device may comprise at least one first suture portion. The device may be configured to arrange the first suture portion within the first snare when the first snare is advanced out of the distal opening of the first needle. Optionally, the device is configured to arrange the first suture portion within the first snare when the first snare is advanced out of the distal opening of the first snare sleeve. The first suture portion may be a first suture end.

The device may further comprise at least one second suture portion. The device may be configured to arrange the second suture portion within the second snare when the second snare is advanced out of the distal opening of the second needle. Optionally, the device is configured to arrange the second suture portion within the second snare when the second snare is advanced out of the distal opening of the second snare sleeve. The second suture portion may be a second suture end.

The first and second suture portions may be formed by a common suture. Optionally, the first and second suture portions are first and second ends of a suture. A common suture may be understood as a suture that includes and connects the first and second suture portions.

The first and/or second suture portion may be configured to remain in the tissue after the device is withdrawn from the tissue opening.

Any of the sutures or suture portions mentioned herein may be made from or comprise a polymeric material (including polylactic acid, polyglycolic acid, polydioxanone, polycaprolactone, polyhydroxybutyrates, as well as copolymers and/or blends thereof), silk, catgut, or others, as known in the art. The sutures or suture portions may be a monofilament or braided. Any of the sutures or suture portions mentioned herein may be made from a resorbable material and/or provided with a coating. Such coating may have antibacterial properties and/or provide for easier gliding. The sutures mentioned herein may be elastically and/or plastically deformable. The sutures mentioned herein may be provided with a glue, one or more growth factors, and/or gelatine. Such glue may be helpful in containing liquid within the body cavity or lumen.

An end of the first and/or second suture portion may be releasably retained proximate a distal end of the shaft. The first and/or second suture portion may be retained in a first configuration proximate the longitudinal axis of the shaft. When released, the end of the first and/or second suture portion projects radially outward from the shaft. When the first and second snares are advanced out of the needles and the first and/or second suture portion is moved from the first to the second configuration, the end of the first and/or second suture portion may be configured to project into the first and second snare, respectively (e.g., into an open loop formed by the first and second snare, respectively). The device may be configured to allow release of the first suture portion only after the first snare is advanced out of the first needle, and optionally out of the first snare sleeve. The device may be configured to allow release of the second suture portion. only after the second snare is advanced out of the second needle, optionally out of the second snare sleeve.

The first and/or second suture portions may be retained in a suture holder provided at a distal portion of the device that is to be inserted into the body cavity or lumen. Optionally, the first and/or second suture portions are configured to be pulled out from the suture holder. Optionally, a length of the first and/or second suture portion extending into the shaft is configured to be pulled in a proximal direction to release the first and/or second suture portions from the suture holder.

The suture holder may be configured to releasably retain a first segment (e.g., the end) of the first and/or second suture portions extending outside a lumen of the shaft. A second segment of the first and/or second suture portions may extend at least partially through the lumen. The lumen may extend into and/or through the shaft, for example, from an opening at the distal end of the shaft. The first and second suture portions may extend into the lumen through a common opening.

The first and/or second suture portion may be configured to be released from the suture holder by pulling the first and/or second suture portion (e.g., said second segment) in the proximal direction.

The distal portion of the device, optionally the distal end of the elongate shaft, may comprise a plurality of grooves along an outer circumferential surface thereof, and the first segments of the first and second suture portions may be received in said grooves. The first segment of the first and/or second suture portions may be pre-mounted in the grooves so that it does not interfere with other moving components of the device (e.g., a basket as described hereinbelow).

The suture holder may be a ring-shaped structure, optionally an elastic ring, optionally wherein the first and/or second suture portions are configured to be at least partially arranged between the ring-shaped structure and the shaft. Alternatively, the suture holder may be formed by a plurality of holes or tubes, optionally wherein the first and/or second suture portions are configured to be at least partially inserted into a respective one of the holes or tubes.

The first and/or second suture portions may be configured to project and/or bend radially outwards when released from the elongate shaft. The stiffness and/or recoil of the suture portions may be selected such that the projecting and/or bending motion provides for a positioning of the suture portions into the snares (e.g., into an open loop of the respective snare when advanced out of the needle). It is believed that forming the first and/or second suture portions from a monofilament may be particularly advantageous for providing the desired stiffness and/or recoil.

The first and/or second suture portions may have a diameter smaller than 0.6 mm, a diameter smaller than 0.4 mm a diameter smaller than 0.25 mm, or a diameter smaller than 0.22 mm.

A segment (e.g., the above-mentioned second segment) of the first and/or second suture portion may be stored within the lumen of the shaft. The segment of the first and/or second suture portions may extend through the lumen along the longitudinal axis. The lumen may extend from the proximal end to the distal end of the elongate shaft. Storage of the segment of the first and/or second suture portion within the lumen of the elongate shaft is believed to allow for a safer and orderly operation as it prevents the suture portions from unintentionally interacting with other components of the device and/or entangling.

The length of each of the first and/or second suture portion stored within the lumen may be at least 30 mm or at least 200 mm. Where the first and second suture portions are formed as a common suture, a length of the suture stored within the lumen may be at least 60 mm or at least 400 mm. Such lengths are believed to be suitable in fetal applications. The lengths may be longer or shorter when closing other tissue openings.

The length of the suture may be stored in the lumen as at least one loop.

The lumen may have a diameter smaller than 5 mm, a diameter smaller than 2 mm, or a diameter smaller than 1 mm. The lumen may have a diameter larger than 0.25 mm or larger than 0.5 mm.

Alternatively or additionally to the lumen, a segment of the first and/or second suture portions may be stored in a cavity and/or a cartridge arranged at the distal end of the elongate shaft. The cavity or cartridge may comprise a spool onto which at least part of the first and/or second suture portions is wound.

The first and/or second suture portions may be coupled to one or more actuators arranged proximate the proximal end of the shaft. The one or more actuators may be configured to pull the first and/or second suture portion in the proximal direction. The one or more actuators may be provided at the handle.

Alternatively, the first and/or second suture portion may extend out of the shaft proximate the proximal end of the shaft to allow the user to grasp the respective portion and pull it proximally by hand.

The device may further comprise one or more washers that are coupled to the first and/or second suture portions and configured to remain in the tissue therewith. The one or more washers may be configured to cover holes created by the first and second needles in the tissue. The one or more washers may have a diameter of less than 3 mm or less than 2 mm, and/or a thickness of less than 2 mm or less than 1 mm. In particular, the one or more washers may be configured to be located within the body cavity or lumen in abutment against the tissue. The one or more washers may be resorbable. Alternatively or additionally, the device may comprise at least one patch configured to remain in the body cavity or lumen and overlap with the closed tissue opening from within the cavity, optionally wherein the patch is elastic and/or resorbable. The patch may be coupled to the first and/or second suture portions.

The tissue, such as the fetal membrane, may be very delicate, and pulling the suture through the tissue or creating a suture knot with too much force may lead to cutting into the tissue. The holes formed by the needles are vulnerable areas within the tissue. Placing one washer, e.g., with a diameter of 1.5 mm and a thickness of 0.5 mm, below the suture at each hole may be helpful in minimizing the risk of cutting into the tissue. Such washer could also help to press the fetal membrane towards the uterus, whereby it is believed (without wanting to be bound by theory) that resistance of the fetal membrane to cutting may be increased.

The tissue may comprise at least one first and at least one second tissue layer, the second tissue layer extending between the first tissue layer and the body cavity or lumen. A third tissue layer (e.g., the fetal membrane) may be attached to the second tissue layer (e.g., the uterus). Instead or in addition to such third tissue layer, an artificial sheath may be positioned in the cavity and attached to the second tissue layer.

The device may further comprise a support structure at the distal end of the elongate shaft. The support structure is configured to move between a first, contracted configuration and a second, expanded configuration. The support structure is configured to be expanded in the body cavity or lumen. The support structure in the second, expanded configuration may be configured to be pressed against the second tissue layer, optionally from within the cavity, in a proximal direction, optionally by pulling the elongate shaft in the proximal direction. The support structure may provide for a more stable positioning of the device and/or may support the second and/or third tissue layer during penetration of the needles.

The first needle may be configured to penetrate through the second tissue layer along a first trajectory, the first trajectory extending from the respective exit opening through the second tissue layer obliquely to the longitudinal axis and radially outwards towards the support structure, and/or the second needle may be configured to penetrate through the second tissue layer along a second trajectory, the second trajectory extending from the respective exit opening through the second tissue layer obliquely to the longitudinal axis and radially outwards towards the support structure.

The support structure may be formed as a support basket, optionally as a nitinol basket.

The device may further comprise at least one expandable pushing member configured to push the first tissue layer out of the first and second trajectories, the pushing member being configured to move between a radially contracted configuration and a radially expanded configuration, optionally wherein the device is configured to move the pushing member from the contracted configuration to the expanded configuration by compressing the pushing member in the longitudinal direction of the shaft.

Optionally, the pushing member is configured to push away the overlaying first tissue layer, e.g., fascia, to prevent the first tissue layer from being penetrated by the needles. Thereby, an undesired stitching that would connect the first tissue layer and the second tissue layer can be avoided, which may otherwise cause medical complications, e.g., in case of a patient during pregnancy, by the uneven expansion of the tissue layers surrounding the womb.

In other words, at least a portion of the first and second trajectories may extend through a cavity created between the pushing member and the elongate shaft when the pushing member is in the expanded configuration. The pushing member in the expanded configuration may form an enclosure and/or cage, wherein at least a portion of the first and second trajectories extends through said enclosure and/or cage. As a result, each of the needles can move within the cavity created between the elongate shaft and the pushing member, or enclosure and/or cage, thereby preventing the contact between the needles and the first tissue layer.

The needle exit openings may be located distal from a first position at which the pushing member is coupled to the elongate shaft, optionally distal from a most proximal position at which the pushing member is coupled to the elongate shaft when the pushing member is expanded. Alternatively or additionally, the exit openings may be located proximal from a second position at which the pushing member is coupled to the elongate shaft, optionally proximal from a most distal position at which the pushing member is coupled to the elongate shaft when the pushing member is expanded.

The at least one pushing member may be configured to not radially extend beyond an outer diameter of the elongate shaft when in the contracted configuration. Furthermore, the pushing member, when expanded, may be configured to lift the first tissue layer away from the second tissue layer in a proximal direction. Alternatively or additionally, the pushing member may be configured to push the first tissue layer away from the device perpendicularly to the longitudinal axis in a radially outward direction. Such motion of the pushing member is believed to be beneficial for lifting the first tissue layer, e.g., fascia, in a soft and safe manner without damaging other tissue layers.

The at least one pushing member may be formed as a pushing member basket, optionally as a nitinol basket. The pushing member basket may be integrally and/or monolithically formed with the support basket forming the support structure. Alternatively or additionally, the pushing member basket and the support basket forming the support structure may be connected by one or more struts extending along the longitudinal axis.

The pushing member may be expandable together with the support structure or independently of the support structure.

The expandable pushing member and the support structure may be combined and/or integrated into a single sub-component and/or into a single expandable sub-assembly of the device. This may provide for a space-saving design, which is particularly advantageous for, e.g., fetoscopy, but the devices disclosed herein are not limited in this regard. Depending on the chosen application, providing the expandable pushing member and the support structure as separate sub-assemblies is also considered. For example, the expandable pushing member and the support structure may be provided at the same or at different positions along the longitudinal axis of the elongate shaft.

The device, optionally the handle, may be configured such that the pushing member can only be moved to its expanded configuration once the support structure is moved to its expanded configuration.

In the expanded configuration, the at least one pushing member may be located at least 2 mm away, optionally at least 5 mm away, and/or at most 100 mm away, optionally at most 20 mm away, from the distal end of the elongate shaft. These distances are believed to be helpful for pushing the relevant tissue, in particular fascia, out of the trajectory of the needles.

The pushing member may comprise a membrane cover, optionally wherein the membrane cover covers at least a proximally facing side of the pushing member.

The at least one pushing member may comprise a plurality of flaps, optionally two or at least four flaps. A proximal portion of each of the flaps may be connected to the elongate shaft at a first position, optionally via a first pushing member collar, optionally wherein the first pushing member collar is stationary with respect to the elongate shaft. A distal portion of each of the flaps may be connected to the elongate shaft at a second position, optionally via a second pushing member collar, optionally wherein the second position is distal from the first position, optionally wherein the second pushing member collar is movable with respect to the elongate shaft along the longitudinal axis. The proximal and distal portion of each flap may be connected via a hinge portion, optionally wherein the flaps bend at the hinge portion when the pushing member is moved to its expanded configuration, optionally wherein the hinge portion has a lower resistance to bending than the proximal and distal portions.

The at least one pushing member, optionally the at least one flap, may comprise at least one marker for determining the configuration of the pushing member in a medical imaging procedure, optionally wherein the marker is an ultrasound marker and/or a radiopaque material. It is preferred that the at least one pushing member, optionally the at least one flap, is made from an ultrasound visible material and/or a radiopaque material.

Ultrasound visibility may be achieved, for example, by using porous polymers (e.g. foams), providing gas bubbles on inner or outer surfaces of the flaps (as discussed, e.g., in WO 2012/148265 A1, which is herein incorporated by reference in its entirety), surface roughening (e.g., by means of polishing, sand blasting or electric discharge machining), or by introducing contrast enhancing particles into the material (as discussed, e.g., in EP 1 155 418 B1, which is herein incorporated by reference in its entirety). It should be noted that any intervention in accordance with the methods of the present invention may be performed under ultrasound guidance. In particular, such ultrasound guidance may be provided during the expansion and/or retraction of the support structure, during the expansion of the at least one pushing member, and/or when advancing the needles towards the expanded support structure. Optionally, the fetal intervention (e.g., fetoscopy) can be performed using an ultrasound guidance throughout the operation or at least during the expansion of the at least one pushing member and when the needles are advanced towards the expanded support structure. The use of an ultrasound visible material may provide additional visibility and certainty whether the first tissue layer has been sufficiently pushed away before the advancement of the needles.

Alternatively or additionally to the above-mentioned flaps, the at least one pushing member may be provided as or may comprise an inflatable structure, for example an inflatable balloon. Such inflatable structure may be used, e.g., to expand the flaps (by inflating the inflatable structure, for example with a gas or a liquid).

The elongate shaft may have a length between 10 mm to 2000 mm, between 50 mm to 500 mm, or between 100 mm to 200 mm. The elongate shaft may be rigid and/or straight along the longitudinal axis. Furthermore, the elongate shaft may have a diameter smaller than 15 mm, a diameter smaller than 7 mm, or a diameter smaller than 4 mm. It is preferred that the elongate shaft is made from a metallic material (such as stainless steel), fibre reinforced polymers, or provided by a polymer tube, optionally an extruded polymer tube.

The handle may be provided at the proximal end of the elongate shaft. The handle may be adapted for allowing the user to hold the device in a manner that is convenient for the respective application. The handle may comprise measurement indicator lines to provide the user with visual information regarding the positioning of the instrument within the cavity or lumen.

The device may further comprise at least one actuator, optionally a plurality of actuators, at the proximal end of the elongate shaft, optionally at the handle. The actuators may be used for operating the expandable pushing member and/or the support structure, for advancing and/or retracting the needles, for advancing and/or retracting a suture, and/or for injecting at least one fluid through the needles.

The device comprises a distal end. The distal end optionally is atraumatic. The distal end may be formed by the distal end of the elongate shaft, which may be atraumatic (e.g., hemispherical or rounded). The atraumatic end of the elongate shaft reduces the risk of potential damages that may be caused by unintentional contact, e.g., with a fetus.

A distal portion of the device, optionally the distal end of the elongate shaft, may comprise a plurality of recesses or openings, wherein each of the recesses or openings may be configured to at least partially receive a respective one of the snares when the snare is advanced out of the respective snare sleeve. Such configuration is believed to allow for a more precise positioning of the snares at the distal end of the elongate shaft.

The distal portion of the device, optionally the distal end of the elongate shaft, may comprise a stop feature for limiting the distal movement of the support structure, optionally wherein the stop feature is a protrusion that extends radially outward from the shaft. The stop feature may prevent the support structure from being inadvertently released from the elongate shaft.

The longitudinal axis of the elongate shaft may be configured to be oriented substantially perpendicular to the tissue when closing the tissue opening.

The needle guides may have a diameter smaller than 2 mm, optionally a diameter smaller than 1 mm and/or a diameter larger than 0.5 mm, optionally a diameter larger than 0.7 mm.

The exit openings may be spaced at least 3 mm, optionally at least 8 mm in the proximal direction from a proximally facing side of the support structure that is pressed against the second tissue when the support structure is in the second, expanded configuration. The exit openings may be located proximal of a position at which the support structure is coupled to the elongate shaft, optionally proximal from a most proximal position at which the support structure is coupled to the elongate shaft.

The size dimensions of the needle guides as described above are considered most appropriate and effective for the use in closing a tissue opening, in particular in fetoscopy.

Each of the needle guides may have a first portion and a second portion, the second portion extending obliquely to the longitudinal axis and/or radially outward, optionally wherein the first portion is substantially parallel to the longitudinal axis. Furthermore, the second portion may form a distal portion of the respective needle guide. It is preferred that each of the needle guides comprises a curved segment, optionally wherein the curved segment is located between the first. portion and the second portion, and/or the second portion is curved. Each of the needle guides may be configured such that the trajectory along which the respective needle extends from the exit opening extends at an angle to the longitudinal axis, optionally wherein the angle is at most 50° or at most 30°, and/or wherein the angle is at least 5° or at least 10°.

The oblique extension of the needle guides may determine the trajectories of the needles from the exit openings towards the support structure. The oblique design may also determine the distance between two opposing needles when the needles reach their intended position at the support structure, which in turn defines the stich pattern and/or the size of tissue opening that may be closed.

Optionally, the needle guides and/or exit openings are spaced around a periphery of the elongate shaft, and, optionally, the exit openings are spaced at regular intervals around the periphery of the elongate shaft.

The exit openings may be located along a peripheral side surface of the elongate shaft. Alternatively, the exit openings may be located at a distally facing surface of the elongate shaft, e.g., at a distally facing surface of the shaft provided by a reduction of the shaft's diameter. The needles may be pre-mounted in their respective needle guides.

Each needle guide may have an entry opening at its proximal end. Optionally, the elongate shaft comprises two or more entry openings, optionally four entry openings. The entry openings may be located along an outer side surface of the elongate shaft. The needles may be pre-mounted in their respective needle guides. The entry openings allow for an easier mounting and adjustment of the needles within the needle guides of the elongate shaft.

The elongate shaft may comprise at least three needle guides, optionally at least four needle guides. The elongate shaft may comprise at least three exit openings, optionally at least four exit openings.

The support structure in the first, contracted configuration may be configured to be inserted through the tissue opening into the body cavity or lumen. For this purpose, the support structure in the first, contracted configuration may be configured to not radially extend beyond an outer diameter of the elongate shaft and/or the support structure in the first, contracted configuration may have a diameter of 10 mm or less or 5 mm or less.

The support structure in the second, expanded configuration may be configured to be pressed against the second tissue layer, optionally from within the cavity in a proximal direction, optionally by pulling the elongate shaft in the proximal direction. For this purpose, the support structure in the second, expanded configuration may extend beyond the outer diameter of the elongate shaft and/or the support structure in the second, expanded configuration may have a diameter of at least 5 mm or at least 8 mm. Optionally, the support structure is configured to switch between the first, contracted configuration and the second, expanded configuration by pushing and pulling the elongate shaft along the longitudinal axis, respectively. In this manner, the device can be easily inserted through the tissue opening and deployed within the body cavity or lumen of the tissue to stably press against the tissue, e.g., against the second tissue layer, from within the cavity. Alternatively, the support structure may be configured to switch between the first, contracted configuration and the second, expanded configuration by moving a proximal end of the support structure along the elongate shaft in the proximal direction and/or by pushing and pulling wires extending through the lumen in the elongate shaft. In other words, the support structure may be configured to be expanded by compressing the support structure along the longitudinal axis.

The support structure may comprise a membrane cover and/or one or more inflatable members. When using an inflatable member, the support structure may be moved from the collapsed configuration to the expanded configuration by inflating the one or more inflatable members. The membrane cover may prevent a portion of the tissue from being caught by the support structure, e.g., when the support structure moves from the expanded configuration back to the contracted configuration.

The support structure may comprise a plurality of support arms such as at least two, at least four, or at least six support arms. Each of the support arms in the first, contracted configuration may be substantially parallel to the longitudinal axis.

A proximal portion of each support arm may be connected to the elongate shaft at a first position, optionally via a proximal collar of the support structure (e.g., a proximal collar of the support basket). The proximal portion of the support arms in the second, expanded configuration may be substantially perpendicular to the longitudinal axis.

A distal portion of each support arm may be connected to the elongated shaft at a second position, optionally via a distal collar of the support structure (e.g., a distal collar of the support basket). The device may be configured to be actuated to move the distal portion and/or the distal collar along the elongate shaft in the proximal direction to move the support structure from the first, contracted configuration to the second, expanded configuration.

The proximal and distal portions of the support arms may be connected via a hinge portion, optionally wherein the support arms bend at the hinge portion when the support structure is moved to its expanded configuration, optionally wherein the hinge portion has a lower resistance to bending than the proximal and distal portions. This configuration may provide for a stable design that is cost-effective to manufacture at the desired sizes.

It is preferred that the proximal and distal portions of the support arms are integrally formed and/or formed as a monolithic component. In particular, the support structure may be formed as a support basket, optionally wherein the support basket is made from nitinol. This provides for a reliable and space-saving arrangement. The support arms may be formed by a plurality of struts of the support basket, optionally wherein the struts are provided with bending and/or hinge portions having a reduced thickness and/or width.

One or more pairs of the support arms may be configured to expand on diametrically opposite sides of the elongate shaft. A support extension may extend radially outward from each support arm when the support structure is in the second, expanded configuration.

When the first and second needles are in the extended position and the support structure is in the second, expanded configuration, the distal opening of the first needle and the distal opening of the second needle may be arranged distally of the first position at which the proximal portion of the support arms is connected to the elongate shaft, optionally distal of the proximal collar, and proximally of the second position at which the distal portion of the support arms is connected to the elongate shaft, optionally proximal of the distal collar.

Additionally or alternatively, when the first and second needles are in the extended position and the support structure is in the second, expanded configuration, the distal opening of the first needle and the distal opening of the second needle may be arranged closer to the longitudinal axis than a radially most-outward portion of the support arms (e.g., closer to the longitudinal axis than the hinge portions).

In other words, when the first and second needles are in the extended position and the support structure is in the second, expanded configuration, the distal opening of the first needle and the distal opening of the second needle may be arranged within an enclosure and/or cage formed by the support structure and/or the support arms.

The support structure, optionally the support arms, may be made from an ultrasound visible material and/or a radiopaque material.

The first needle and the second needle may be configured to move separately or to move jointly upon actuation of an actuator of the device.

The first needle and/or the second needle may have a sharp distal end, optionally wherein the distal end has a dull and a sharp portion, optionally wherein the dull portion is provided by a chamfered or radiused edge section. Optionally, the dull portion is located closer to the elongate shaft than the sharp portion or more inward. Such dull portion may prevent the snare from being cut by the respective needle during retraction of the snare into the needle.

The at least one first needle and/or the at least one second needle may comprise a proximal portion and a distal portion, wherein the distal portion is more flexible than the proximal portion. For this purpose, the at least one first needle and/or the at least one second needle may comprise a distal portion made from nitinol and/or a proximal portion made from stainless steel, nitinol or titanium. Alternatively or additionally, the at least one first needle and/or the at least one second needle may comprise a bending portion, optionally wherein a sidewall of the first and/or second needle is provided with a plurality of slots and/or struts along the bending portion, optionally wherein the slots are made by laser cutting.

The first needle and/or the second needle may have an outer diameter of 0.65 mm to 0.8 mm, and/or an inner diameter of 0.5 mm to 0.65 mm. Such size dimensions are believed to be most appropriate for suturing the fetal membrane.

The device may be configured to inject a fluid through the at least one first needle and/or the at least one second needle, optionally wherein the fluid contains a growth factor, an anesthetic, and/or a glue. Since the stitching holes can be vulnerable areas that can possibly tear in the closure of the tissue opening, additionally injecting fluids, e.g., glue or growth factor, can stabilize the tissue.

It is preferred that the device comprises at least two first needles and at least two second needles. Four needles may be advantageous for providing a cross-stitching with two sutures. A cross-stitching is believed to be beneficial for ensuring an adequate closure of the tissue opening in view of the small dimensions of the opening, e.g., in fetoscopy.

The distal end of the first and second snare sleeves may be provided with one or more notches. The first and/or second snare sleeve may be a polymeric tube. The first snare may be configured to be closed by advancing the first snare sleeve distally out of the first needle towards the elongate shaft and/or the second snare may be configured to be closed by advancing the second snare sleeve distally out of the second needle towards the elongate shaft.

According to a further aspect, the invention relates to a method of suturing tissue, in particular a tissue opening. The method comprises suturing the opening using any of the devices described herein. In particular, the method may comprise inserting an elongate shaft through the tissue opening into a bodily cavity or lumen. Optionally, a support structure may be opened in the bodily cavity or lumen. First and second hollow needles may be advanced out of respective needle guides in a distal and/or radially outward direction from a retracted position to an advanced position. First and second snares may be advanced out of distal openings of the first and second hollow needles, respectively, wherein the snares may bend in a radially inward direction towards the elongate shaft when advanced. First and second suture portions held at the elongate shaft may be released to bend radially outwards into the advanced snares (e.g., by pulling the suture portions out of a suture holder). The snares may then be closed with the respective suture portion being held therein, e.g., by retracting the snare into the respective needle and/or by advancing a snare sleeve over the respective snare. The closed snares may then be retracted into and through the needles in a proximal direction with the suture portions retained therein. The needles may then be retracted, the support structure may be closed, and the elongate shaft may be withdrawn from the tissue opening.

The present summary is provided only by way of example and not limitation. Other aspects of the present invention will be appreciated in view of the entirety of the present disclosure, including the entire text, claims, and accompanying figures.

### BRIEF DESCRIPTION OF THE DRAWINGS

The invention will be described in more detail with reference to the figures below. These figures disclose embodiments of the invention for illustrational purposes only. In particular, the disclosure provided by the figures is not meant to limit the scope of protection conferred by the invention.

FIG. 1 is a schematic front view of a device for closing a tissue opening in accordance with an embodiment of the present invention.

FIGS. 2A-2F are schematic perspective views illustrating a distal end of the device of FIG. 1 during individual procedural steps performed for the closure of a tissue opening.

FIG. 3A is a schematic front view illustrating the distal end of the device of FIG. 1 while inserted into a tissue opening and shifted to an expanded configuration. First and second needles, first and second snare sleeves, and first and second snares are each shown in a distally advanced configuration.

FIG. 3B is a schematic bottom view illustrating the device of FIG. 3A.

FIG. 4A is a schematic cross-sectional perspective view showing a proximal portion of the device of FIG. 1.

FIG. 4B is a schematic cross-sectional perspective view showing a distal portion of the device of FIG. 1.

FIG. 5 is a schematic perspective view of a second tissue layer of the tissue with sutures and washers according to the present invention, as seen from within a body cavity or lumen.

### DETAILED DESCRIPTION

As used herein, “proximal” means closer/closest to the surgeon, while “distal” means further/farthest from the surgeon. In other words, “proximal” means further/farthest from the tissue opening, while “distal” means closer/closest to the tissue opening, and/or farthest within the body cavity or lumen (i.e., for those parts of the device inserted into the cavity or lumen).

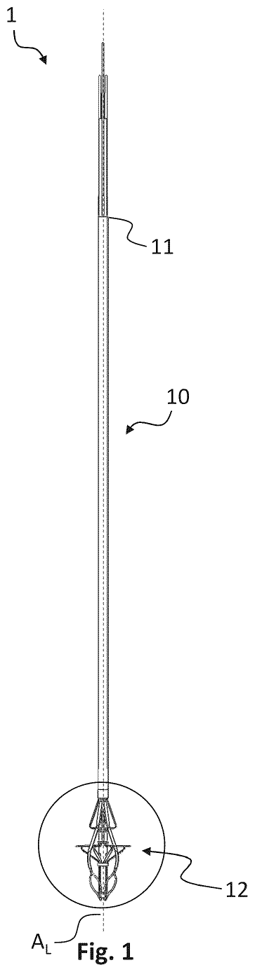

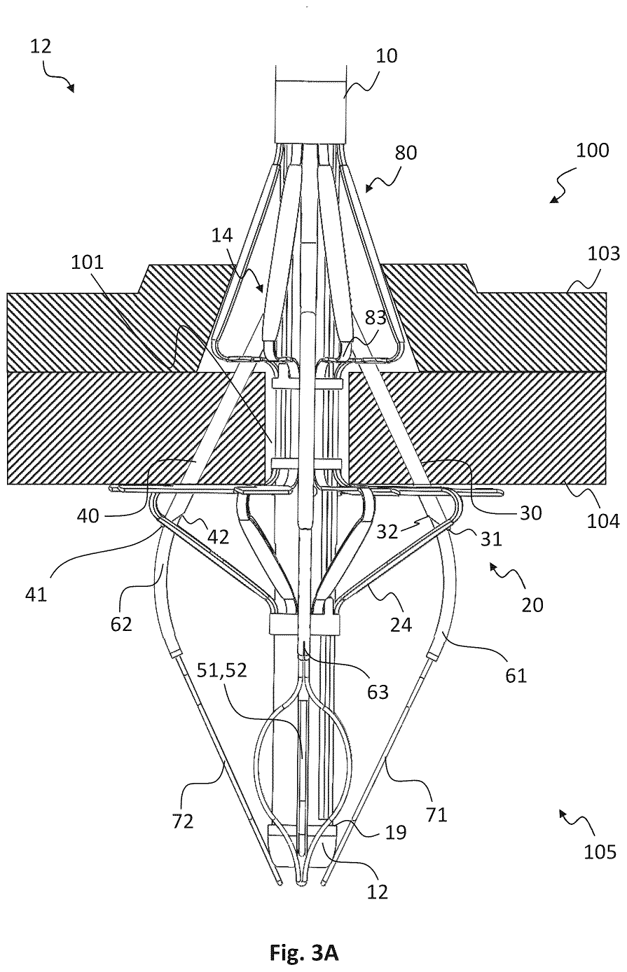

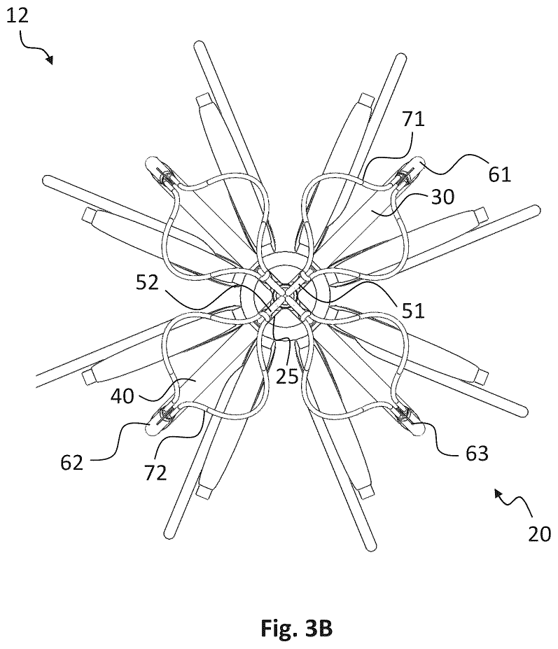

FIG. 1 schematically illustrates a device 1 according to the invention. The device 1 comprises an elongate shaft 10 having a proximal end 11, a distal end 12 and extending along a longitudinal axis A_{L}. The proximal end 11 may comprise a handle (not illustrated) to be operated by the user. The distal end 12 is configured to be inserted into a body cavity or lumen 105 of a tissue 100 through a tissue opening 101 in the tissue 100 (see FIG. 3A).

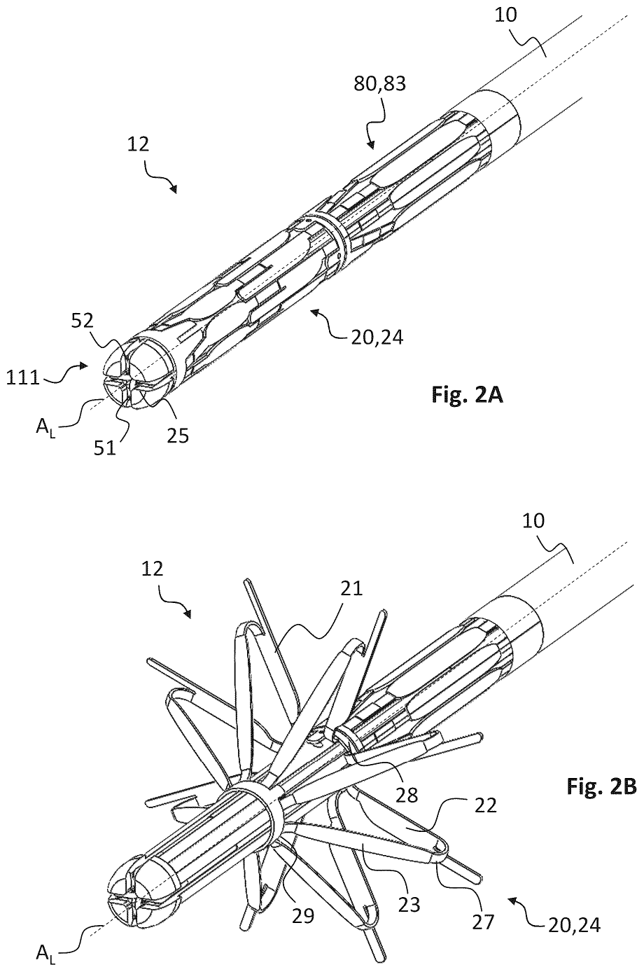

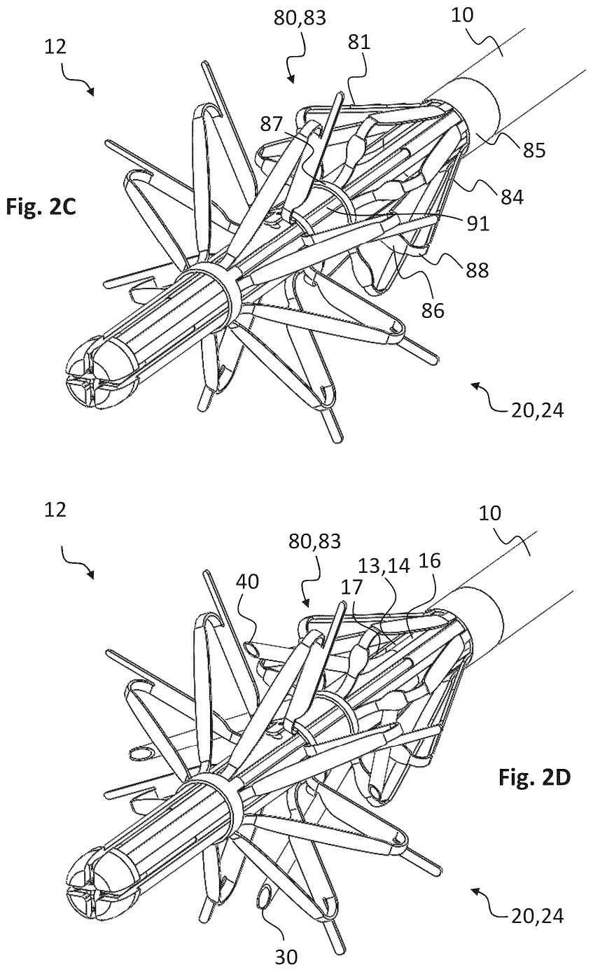

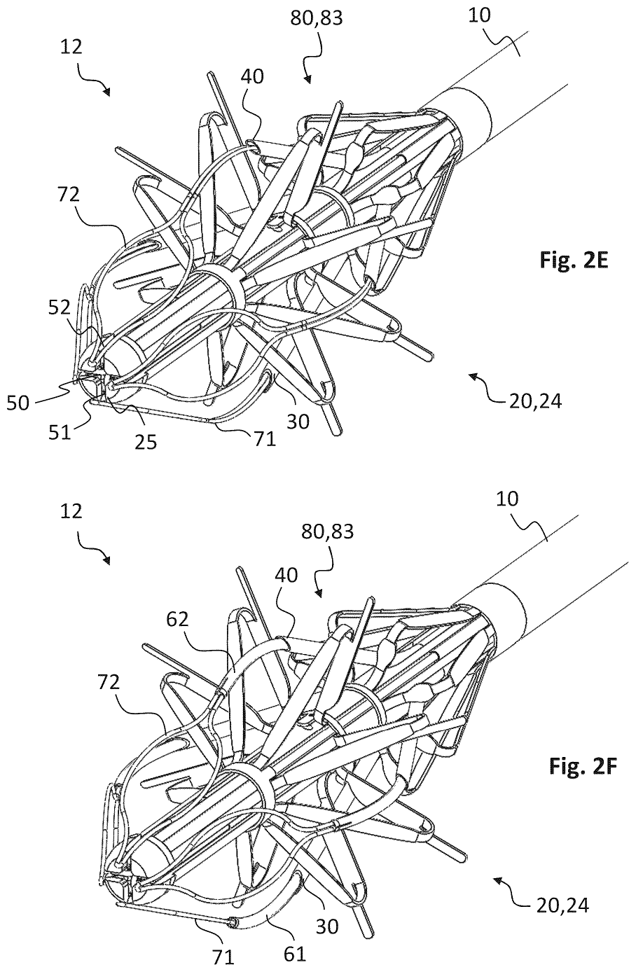

FIGS. 2A-2F and 3A and 3B show a detailed view of the distal end 12 of the device 1, as indicated by the circled area in FIG. 1. As shown, the device 1 may comprise an atraumatic distal tip 111 to avoid injuring the body cavity or lumen (or a fetus in said body cavity).

FIGS. 2A-2F schematically illustrate the device 1 being used for closing the tissue opening 101 in the tissue 100.

After the desired surgery and/or an endoscopic procedure is completed, the related instruments are removed. A catheter (not illustrated) providing access to the tissue opening 101 may remain inserted in the tissue opening 101.

FIG. 2A illustrates the device 1 in a configuration in which said device 1 may be inserted through the catheter, so that the distal end 12 of the device 1 protrudes into the body cavity or lumen.

As shown in FIG. 2A, a groove or channel 25 may be formed at the distal end 12, in particular at the distal tip 111. As shown, a plurality of grooves 25 may extend along the distal end 12 of the device 1 along its outer circumferential surface. The grooves 25 are configured to receive segments of first and second suture portions 51, 52 in the first, contracted configuration. The segments of the first and second suture portions 51, 52 are released from the grooves 25 when the device 10 is in the second, expanded configuration, as described in more detail below. The first and second suture portions 51, 52 may be first and second ends of a suture 50.

In a next step, as shown in FIG. 2B, a support structure 20 at the distal end 12 is shifted from a first, contracted configuration to a second, expanded configuration, e.g., by pulling an actuator or by compressing the support structure 20 along the longitudinal axis A_{L}. Once expanded, the support structure 20 may be pulled proximally against the tissue 100, e.g., against a second tissue layer 104 (see FIG. 3A), which can be a uterus. The second tissue layer 104 extends between a first tissue layer 103 and the body cavity or lumen 105. The first tissue layer 103 may be fascia. As shown in FIG. 3A, the first tissue layer 103 may be lifted and/or pushed away from the second tissue layer 104. The skilled person will appreciate that the first and second tissue layers 103, 104 are schematically illustrated in FIG. 3A with an angular shape. It will be understood, however, that the shape of said tissue layers will be more organic and rounder in reality.

Turning back to FIG. 2B, the support structure 20 comprises multiple support arms 21. A proximal portion 22 of each support arm 21 is connected to the elongate shaft 10 via a proximal collar 28 of the support structure 20. A distal portion 23 of each support arm 21 is connected to the elongate shaft 10 via a distal collar 29 of the support structure 20. The support structure 20 is formed as a support basket 24 with the support arms 21 being formed by a plurality of struts.

The support structure 20 is expanded by approaching the distal and proximal collars 28, 29 to one another, e.g., by moving the distal collar 29 in the proximal direction along the elongate shaft 10. This leads to bending of a hinge portion 27 that connects the proximal and distal portions 22, 23 and optionally has a lower resistance to bending than these two portions 22, 23. The support structure 20 is thereby moved between the first, contracted configuration to the second, expanded configuration.

In the first, contracted configuration, each of the support arms 21 is configured to be substantially parallel to the longitudinal axis A_{L}, whereas in the second, expanded configuration, the proximal portion 22 of the support arms 21 is configured to be substantially perpendicular to the longitudinal axis (A_{L}).

After or during expansion of the support structure 20, an expandable pushing member 80 is deployed from a first, contracted configuration to a second, expanded configuration by using an actuator (see FIG. 2C). The expandable pushing member 80 lifts and/or pushes the first tissue layer 103 that is located proximally from the second tissue layer 104 away from the trajectories of the first and second needles 30, 40 (see again FIG. 3A).

The expandable pushing member 80 comprises a plurality of flaps or pushing arms 81. A proximal portion 84 of the arms 81 is connected to the elongate shaft 10 via a first pushing member collar 85. A distal portion 86 of the arms 81 is each connected to the elongate shaft 10 via a second pushing member collar 87. The pushing member 80 is expanded by approaching the distal and proximal collars 85, 87 to one another, e.g., by moving the distal collar 87 in the proximal direction along the elongate shaft 10. The first pushing member collar 85 may be stationary with respect to the elongate shaft 10.

A hinge portion 88 that connects the proximal portion 84 and the distal portion 86, and optionally has a lower resistance to bending than these two portions 84, 86, bends when the expandable pushing member 80 is moved from the first, contracted configuration to the second, expanded configuration.

The pushing member 80 and the support structure 20 can be provided as a unitary expandable structure, as shown. The pushing member 80 and the support structure 20 may be connected to each other by one or more struts of said structure that extend along the longitudinal axis A_{L}. The unitary expandable structure may be a basket made of metal or polymeric materials, e.g., nitinol. Optionally, the support arms 21 and the pushing arms 81 are formed by laser cutting a thin tubular body.

FIG. 2D illustrates how the first and second needles 30, 40 are advanced out from exit openings 14 of the elongate shaft 10 (e.g., through the second tissue layer 104; cf. FIG. 3A) towards the support structure 20. Four needles 30, 40 may be employed for providing a cross-stitch.

The exit openings 14 are positioned at the distal end of needle guides 13 that extend along the elongate shaft 10. The exit openings 14 may be positioned in a space encaged and/or enclosed by the pushing member 80 when said pushing member 80 is expanded. This is believed to ensure that the trajectory of the needles 30, 40 will be free from the first tissue layer 103.

The needle guides 13 have a first portion 16 and a second portion 17. The second portion 17 forms a distal portion of the respective needle guides 13 and extends obliquely to the longitudinal axis AL of the elongate shaft 10. As a result, the trajectories of the needles 30, 40 extend obliquely to the longitudinal axis AL of the elongate shaft 10, and optionally in a radially outward direction.

The needles 30, 40 are pushed out from the needle guides 13 in a distal and radially outward direction within a first enclosure formed by the expanded pushing member 80. Optionally, the first and second needles 30, 40 puncture only through the second tissue layer 104, and reach a distalmost position (see also FIG. 3A) within or proximate a second enclosure and/or cage formed by the support structure 20. Optionally, the distal end of the needles 30, 40 does not protrude out of the space enclosed by the expanded support structure 20. In other words, the distal end of the needles 30, 40 in its distalmost position optionally is arranged within the space enclosed by the support structure 20. This is believed to be helpful for preventing an undesired contact between the needles 30, 40 and another tissue inside the body cavity or lumen 105 (e.g., a fetus).

The first and/or second needles 30, 40 are hollow. They may comprise a needle lumen extending therethrough. The needle lumen may be configured for advancing and/or retracting the respective snare and/or the respective snare sleeve therethrough. The needle lumen may also be configured to inject fluid, such as a fluid with a growth factor or a glue, into the tissue 100.

When the device 1 is in the position shown in FIG. 2D, at least one first snare 71 and at least one second snare 72 are advanced out of the hollow needles 30, 40, respectively, towards the distal end of the elongate shaft 10. As shown, the snares 71, 72 may be pre-shaped (e.g., via a heat treatment) to bend towards the elongate shaft when extended out of the needles 30, 40. It is believed that such pre-shaped snares are helpful for ensuring that the first and second suture portions 51, 52, when released from the elongate shaft 10, are captured in the snares 71, 72. However, other configurations are also envisaged, which may be suitable depending on the size of the device, the type of tissue opening, and the mechanical characteristics of the suture.

Once the pre-shaped snares 71, 72 are arranged in proximity of the distal end of the elongate shaft 10 as shown in FIG. 2E, the first and second suture portions 51, 52 that are pre-mounted and releasably retained proximate the distal end 111 of the elongate shaft 10 are released (not illustrated). Said first and second suture portions 51, 52, when released, recoil into the open loops of the respective snare 71, 72. Various different release mechanisms can be implemented for this purpose.

Subsequently, as shown in FIG. 2F, at least one first snare sleeve 61 and at least one second snare sleeve 62 are advanced from the first needle 30 and the second needle 40, respectively. By advancing the snare sleeves distally, the loops of the snares 71, 72 can be made narrower and closed (not illustrated), thereby capturing the first and second suture portions 51, 52 with the first and second snares 71, 72, respectively.

The sutures portions 51, 52 and the first and second snares 71, 72 are then retracted proximally into the first and second needles 30, 40, and pulled in a proximal direction therethrough. The needles 30, 40 are then retracted into the shaft 10 and the support structure 20 is moved back to its contracted configuration. The device 10 is then retracted proximally from the tissue opening 101. While or after removing the device 10 from the opening 101, the suture portions 51, 52 may be released, thereby leaving the suture portions 51, 52 threaded through the second tissue layer 104 but accessible from outside the body cavity or lumen 105. The tissue opening 101 may then be closed by pulling the edges of the opening 101 towards each other, e.g., by tying the suture portions 51, 52 together with a knot. Where four needles 30, 40 are used, the sutures may form two half loops, each of which traverses the tissue opening 101 (e.g., forming a cross-stitch).

FIG. 3A schematically illustrates the device 1 in its fully expanded configuration, as also shown in FIG. 2F. FIG. 3B schematically illustrates the device 1 in the configuration of FIG. 3A from below. FIGS. 3A and 3B illustrate both the support structure 20 and the expandable pushing member 80 in their expanded configuration. The first and second needles 30, 40 are fully advanced into their distalmost position, penetrating only through the second tissue layer 104.

After the first and second snares 71, 72 are advanced out into a position in proximity of the distal end of the elongate shaft 10, the first and second suture portions 51, 52 are released (not illustrated) into the loops of the first and second snares 71, 72, respectively. By advancing the first and second snare sleeves 61, 62, the loops of the snares 71, 72 are closed.

The support structure 20 and/or the pushing member 80 may be made from or comprise a material that has a high ultrasound visibility and/or is radiopaque. This may help the user to determine if the support structure 20 has been adequately expanded distally of the tissue opening 101. Furthermore, this allows a user to see whether the second tissue layer 103 has been adequately moved. For example, an ultrasound and/or radiopaque marker may be provided on the support arms 21 and/or the pushing arms 81. Alternatively, the pushing arms 81 may be made from or coated with an ultrasound visible material and/or a radiopaque material, as described above.

As shown in FIG. 3A, the needles 30, 40 may be provided with a sharper portion 31, 41 and a chamfered and/or duller portion 32, 42 around their distal end. The sharper portion 31, 41 may be optimized for piercing the tissue 100. The duller portion 32, 42 may be provided to avoid damaging the sleeves 61, 62 and/or the first and second suture portions 51, 52 during retraction thereof into the needles 30, 40.

As further shown in FIGS. 3A and 3B, each of the sleeves 61, 62 may be provided with one or more notches 63 at their distal end. In this manner, the sleeves 61, 62 may be configured to better protect the first and second suture portions 51, 52 during retraction into the needles 30, 40.

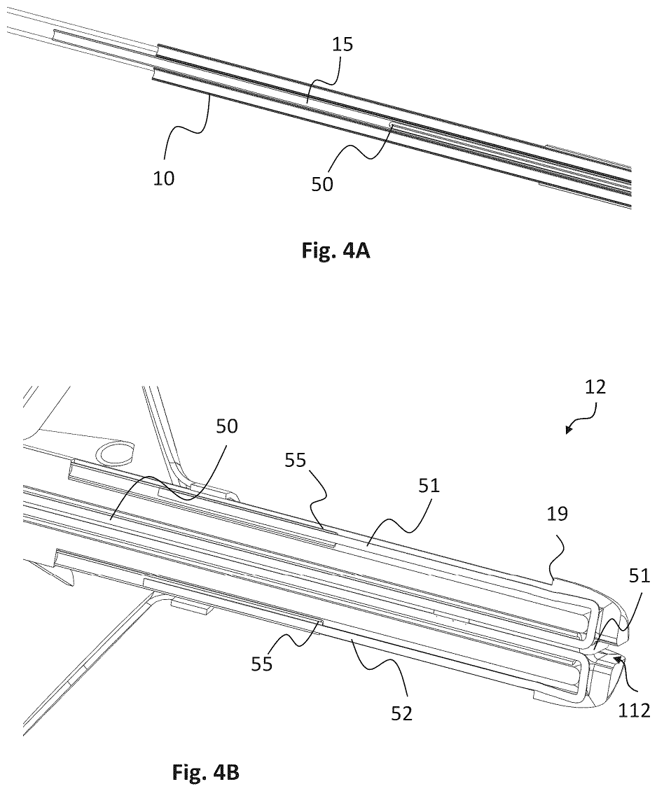

FIG. 4A illustrates a cross-section of the elongate shaft 10 along a section thereof. As shown, a length of suture 50 that connects the first and second suture portions 51, 52 may be stored within a lumen 15 of the elongate shaft 10 (e.g., as a loop). The first and second suture portions 51, 52 may be a first and second end of such common suture 50. One or more such sutures 50 may be stored in the lumen 15 (e.g., two sutures when four needles are used).

The lumen 15 may extend along the longitudinal axis AL of the elongate shaft 10. The elongate shaft 10 comprises at least one distal opening 112 through which the suture 50 may enter into the lumen 15 (see FIG. 4B).

FIG. 4B illustrates a cross-section of the elongate shaft 10 at its distal end 12 with two pre-mounted sutures 50.

As further shown in FIG. 4B, a stop feature 19 can be formed as a protrusion at the distal end 12 of the elongate shaft 10. The stop feature 19 may be configured to limit the distal movement of the support structure 20.

As further shown in FIG. 4B, the first and second suture portions 51, 52 of the suture 50 are releasably retained in one or more suture holders 55. Any suitable structure may be used. For example, such suture holder(s) 55 may be provided by openings or tubes into which a first segment of each of the first and second suture portions 51, 52 is inserted, or by a structure (e.g., a ring) under which the first and second suture portions 51, 52 are clamped to the shaft 10.

The first and second suture portions 51, 52 may be released from the suture holder(s) 55 in any suitable manner. For example, the first and second suture portions 51, 52 may be pulled out of the suture holder(s) 55, e.g., by pulling a second segment of the first and second suture portions 51, 52 that is arranged in the lumen 15 (see FIG. 4A) in the proximal direction (either manually or via an actuator, e.g., an actuator at the handle).

Alternatively or additionally, the support structure(s) 55 may be moved (e.g., via an actuator, e.g., an actuator at the handle). For example, the support structure(s) 55 may be moved in a proximal or distal direction via such actuator, thereby releasing the first segment of the suture portions 51, 52.

Once released from the elongate shaft 10, the first and second suture portions 51, 52 bend and/or recoil radially outwards and into the open loops of the snares 71, 72.

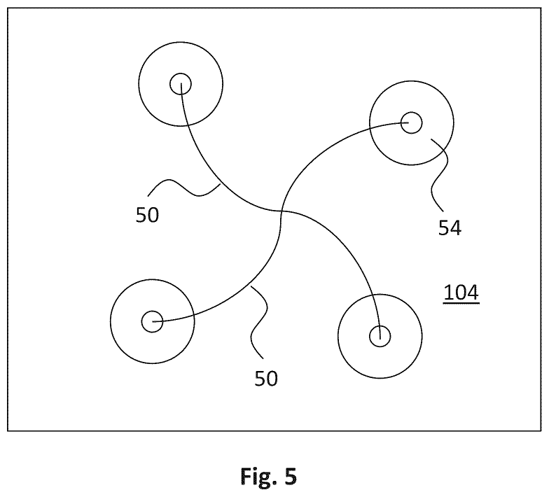

FIG. 5 schematically illustrates two sutures 50 with washers 54 covering the stitching holes in the second tissue layer 104 penetrated by the first and second needles 30, 40. The washers 54 are arranged on the sutures 50 in such manner that they are located within the body cavity 105 after the tissue opening 101 is closed, in abutment against the second tissue layer 104. Alternatively or additionally, at least one patch (not illustrated) that is configured to remain in the body cavity 105 and overlap with the closed tissue opening 101 from within the cavity 105 may be used.

The invention may be defined, for example, by the following aspects:
1. A device (1) for closing a tissue opening (101) in a tissue (100), the tissue extending at least partially around a body cavity or lumen (105), the device (1) comprising:
   an elongate shaft (10) having a proximal end (11), a distal end (12) and extending along a longitudinal axis (A_{L}), the elongate shaft (10) configured to be inserted into the tissue opening (101) in the tissue (100), the elongate shaft (10) comprising at least one first and at least one second needle guide (13), each needle guide (13) having an exit opening (14) at its distal end;
   at least one first hollow needle (30) and at least one second hollow needle (40), each of the first and second needles (30, 40) being configured to be translated along and advanced out of a respective one of the needle guides (13) in a distal and radially outward direction from a retracted position to an extended position; and
   at least one first snare (71) configured to be advanced out of a distal opening of the at least one first hollow needle (30) and at least one second snare (72) configured to be advanced out of a distal opening of the at least one second hollow needle (40).
2. The device according to the preceding aspect, wherein the at least one first snare (71) is configured to be shaped to extend in a radially inward direction towards the elongate shaft (10) when advanced out of a distal opening of the at least one first hollow needle (30), and/or wherein the at least one second snare (72) is configured to be shaped to extend in a radially inward direction towards the elongate shaft (10) when advanced out of a distal opening of the at least one second hollow needle (40).
3. The device of any of the preceding two aspects, wherein the device further comprises:
   at least one first snare sleeve (61) configured to be advanced out of a distal opening of the first needle (30); and
   at least one second snare sleeve (62) configured to be advanced out of a distal opening of the second needle (40).
4. The device of any of the preceding three aspects, wherein the device (1) further comprises at least one first suture portion (51) and is configured to arrange the first suture portion (51) within the first snare (71) when the first snare (71) is advanced out of the distal opening of the first needle (30), optionally wherein the first suture portion (51) is a first suture end, and
   at least one second suture portion (52) and is configured to arrange the second suture portion (52) within the second snare (72) when the second snare (72) is advanced out of the distal opening of the second needle (40), optionally wherein the second suture portion (52) is a second suture end.
5. The device according to the preceding aspect, wherein the first and second suture portions (51, 52) are formed as a common suture (50), optionally wherein the first and second suture portions (51, 52) are first and second ends of the suture (50).
6. The device according to any of the preceding two aspects, wherein the first and/or second suture portions (51, 52) are releasably retained proximate a distal end of the shaft (10).
7. The device according to the preceding aspect, wherein the first and/or second suture portions (51, 52) are releasably retained in at least one suture holder (55), optionally:
   wherein the first and/or second suture portions (51, 52) are configured to be pulled out from the suture holder (55), optionally wherein a length of the first and/or second suture portions (51, 52) extending into the shaft (10) is configured to be pulled in a proximal direction to release the first and/or second suture portions (51, 52) from the suture holder (55); and/or
   wherein the device (1) is configured to allow release of the first suture portion (51) only after the first snare (71) is advanced out of the first needle (30) and/or of the second suture portion (52) only after the second snare (72) is advanced out of the second needle (40).
8. The device (1) according to any of the preceding four aspects, wherein a distal portion of the device (1) that is to be inserted into the body cavity or lumen (105) comprises at least one suture holder (55), wherein the suture holder (55) is configured to releasably retain the first and/or second suture portions (51, 52), optionally a first segment of the first and/or second suture portions (51, 52) extending outside a lumen (15) of the shaft (10).
9. The device (1) according to the preceding aspect, wherein a segment, optionally a second segment, of the first and/or second suture portions (51, 52) extends at least partially through the lumen (15), and wherein the first and/or second suture portions (51, 52) are configured to be released from the suture holder (55) by pulling said segment in the proximal direction.
10. The device (1) according to any of the preceding two aspects, wherein the distal portion of the device (1), optionally the distal end (12) of the elongate shaft (10), comprises a plurality of grooves (25) along an outer circumferential surface thereof, and wherein the first segments of the first and second suture portions (51, 52) are received in said grooves (25).
11. The device (1) according to any of the preceding three aspects,
   wherein the suture holder (55) is a ring-shaped structure, optionally an elastic ring, optionally wherein the first and/or second suture portions (51, 52) are configured to be at least partially arranged between the ring-shaped structure and the shaft (10), or
   wherein the suture holder (55) is formed by a plurality of openings or tubes (55b), optionally wherein the first and/or second suture portions (51, 52) are configured to be at least partially inserted into a respective one of the openings or tubes (55b).
12. The device according to any of aspects 6 to 11, wherein the first and/or second suture portions (51, 52) are configured to project and/or bend radially outwards when released from the elongate shaft (10).
13. The device (1) according to any one of aspects 4 to 12, wherein the first and/or second suture portions (51, 52) are a monofilament.
14. The device (1) according to any one of aspects 4 to 13, wherein the first and/or second suture portions (51, 52) have a diameter smaller than 0.6 mm, optionally a diameter smaller than 0.4 mm, optionally a diameter smaller than 0.25 mm, optionally a diameter smaller than 0.22 mm.
15. The device (1) according to any one of aspects 4 to 14, wherein a segment of the first and/or second suture portions (51, 52) is stored within a lumen (15) of the shaft.
16. The device (1) according to the preceding aspect,
   wherein the length of each of the first and/or second suture portions (51, 52) stored within the lumen (15) is at least 30 mm, optionally at least 200 mm,
   optionally wherein the first and second suture portions (51, 52) are formed as a common suture (50) and a length of the suture (50) stored within the lumen (15) is at least 60 mm, optionally at least 400 mm.
17. The device (1) according to any of the preceding two aspects, wherein the segment of the first and/or second suture portions (51, 52) extends through the lumen (15) along the longitudinal axis (A_{L}).
18. The device (1) according to any one of aspects 4 to 17, wherein the elongate shaft (10) comprises at least one distal opening through which the first and/or second suture portions (51, 52) enter into a lumen (15) of the shaft.
19. The device (1) according to any one of the preceding four aspects, wherein the lumen (15) has a diameter smaller than 5 mm, optionally a diameter smaller than 2 mm, optionally a diameter smaller than 1 mm.
20. The device (1) according to any one of the preceding five aspects, wherein the lumen (15) has a diameter larger than 0.25 mm, optionally a diameter larger than 0.5 mm.
21. The device (1) according to any of the preceding six aspects, wherein the lumen (15) extends from the proximal end (11) to the distal end (12) of the elongate shaft (10).
22. The device (1) according to any one of aspects 4 to 21, wherein a segment of the first and/or second suture portions (51, 52) is stored in a cavity and/or a cartridge arranged at the distal end of the elongate shaft (10), optionally wherein the cavity or cartridge comprises a spool onto which at least part of the first and/or second suture portions (51, 52) is wound.
23. The device (1) according to any one of aspects 4 to 22,
   wherein the first and/or second suture portions (51, 52) are coupled to an actuator arranged proximate the proximal end (11) of the shaft (10), optionally wherein the actuator is configured to pull the first and/or second suture portions (51, 52) in the proximal direction; or
   wherein the first and/or second suture portions (51, 52) extend out of the shaft (10) proximate the proximal end (11) of the shaft (10).
24. The device (1) according to any one of aspects 4 to 23, wherein the device (1) further comprises one or more washers (54) that are coupled to the first and/or second suture portions (51, 52) and configured to remain in the tissue (100) therewith, optionally wherein the one or more washers (54) are configured to cover holes created by the first and second needles (30; 40) in the tissue (100).
25. The device (1) according to the preceding aspect, wherein the one or more washers (54) have a diameter of less than 3 mm, optionally less than 2 mm, and/or wherein the one or more washers (54) have a thickness of less than 2 mm, optionally less than 1 mm.
26. The device (1) according to any one of aspects 4 to 25, wherein the device (1) further comprises at least one patch configured to remain in the body cavity or lumen (105) and overlap with the closed tissue opening (101) from within the cavity (105), optionally
   wherein the patch is elastic and/or resorbable; and/or
   wherein the patch is coupled to the first and/or second suture portions (51, 52).
27. The device (1) according to any one of the preceding aspects, wherein the tissue comprises at least first and second tissue layers (103, 104), the second tissue layer (104) extending between the first tissue layer (103) and the body cavity or lumen (105).
28. The device according to any one of the preceding aspects, wherein the device (1) further comprises a support structure (20) at the distal end (12) of the elongate shaft (10) configured to move between a first, contracted configuration and a second, expanded configuration the support structure (20) being configured to be expanded in the body cavity or lumen (105).
29. The device (1) according to the preceding aspect, wherein the support structure (20) in the second, expanded configuration is configured to be pressed against the second tissue layer (104), optionally from within the cavity (105) in a proximal direction, optionally by pulling the elongate shaft (10) in the proximal direction.
30. The device (1) according to any of the two preceding aspects, wherein the first needle (30) is configured to penetrate through the second tissue layer (104) along a first trajectory, the first trajectory extending from the respective exit opening (14) through the second tissue layer (104) obliquely to the longitudinal axis (A_{L}) and radially outwards towards the support structure (20), and the second needle (40) is configured to penetrate through the second tissue layer (104) along a second trajectory, the second trajectory extending from the respective exit opening (14) through the second tissue layer (104) obliquely to the longitudinal axis (A_{L}) and radially outwards towards the support structure (20).
31. The device (1) according to the preceding aspect, wherein the device (1) further comprises at least one expandable pushing member (80) configured to push the first tissue layer (103) out of the first and second trajectories, the pushing member (80) being configured to move between a radially contracted configuration and a radially expanded configuration, optionally wherein the device (1) is configured to move the pushing member (80) from the contracted configuration to the expanded configuration by compressing the pushing member in the longitudinal direction of the shaft (10).
32. The device (1) according to the preceding aspect, wherein at least a portion of the first and second trajectories extends through a cavity created between the pushing member (80) and the elongate shaft (10) when the pushing member (80) is in the expanded configuration.
33. The device (1) according to any of the preceding two aspects, wherein the pushing member (80) in the expanded configuration forms an enclosure and/or cage, wherein at least a portion of the first and second trajectories extends through said enclosure and/or cage.
34. The device (1) according to any of the preceding three aspects,
   wherein the needle exit openings (14) are located distal from a first position at which the pushing member (80) is coupled to the elongate shaft (10), optionally distal from a most proximal position at which the pushing member (80) is coupled to the elongate shaft (10) when the pushing member (80) is expanded; and/or
   wherein the exit openings (14) are located proximal from a second position at which the pushing member (80) is coupled to the elongate shaft (10), optionally proximal from a most distal position at which the pushing member (80) is coupled to the elongate shaft (10) when the pushing member (80) is expanded.
35. The device (1) according to any one of the preceding four aspects,
   wherein the at least one pushing member (80) in the contracted configuration does not radially extend beyond an outer diameter of the elongate shaft (10).
36. The device (1) according to any one of the preceding five aspects,
   wherein the pushing member (80) when expanded is configured to lift the first tissue layer (103) away from the second tissue layer (104) in a proximal direction; and/or
   wherein the pushing member (80) is configured to push the first tissue layer (103) away from the device (1) perpendicularly to the longitudinal axis (A_{L}) in a radially outward direction.
37. The device (1) according to any one of the preceding six aspects,
   wherein the at least one pushing member (80) is formed as a pushing member basket (83), optionally wherein the pushing member basket (83) is a nitinol basket.
38. The device (1) according to the preceding aspect,
   wherein the pushing member basket (83) is integrally and/or monolithically formed with a support basket (24) forming the support structure (20); and/or
   wherein the pushing member basket (83) and a support basket (24) forming the support structure (24) are connected by one or more struts (91) extending along the longitudinal axis (A_{L}).
39. The device (1) according to any one of the preceding eight aspects,
   wherein the pushing member (80) is expandable together with the support structure (20); or
   wherein the pushing member (80) is expandable independently of the support structure (20).
40. The device (1) according to any one of the preceding nine aspects, wherein the device (1), optionally a handle thereof, is configured such that the pushing member (80) can only be moved to its expanded configuration once the support structure (20) is moved to its expanded configuration.
41. The device (1) according to any one of the preceding ten aspects, wherein, in the expanded configuration,
   the at least one pushing member (80) is located at least 2 mm away, optionally at least 5 mm away, from the distal end (12) of the elongate shaft (10); and/or
   the at least one pushing member (80) is located at most 100 mm away, optionally at most 20 mm away, from the distal end (12) of the elongate shaft (10).
42. The device (1) according to any one of the preceding eleven aspects, wherein the pushing member (80) comprises a membrane cover, optionally wherein the membrane cover covers at least a proximally facing side of the pushing member (80).
43. The device (1) according to any one of the preceding twelve aspects, wherein the at least one pushing member (80) comprises a plurality of flaps (81), optionally two or at least four flaps (81).
44. The device (1) according to the preceding aspect, wherein a proximal portion (84) of each of the flaps (81) is connected to the elongate shaft (10) at a first position, optionally via a first pushing member collar (85), optionally wherein the first pushing member collar (85) is stationary with respect to the elongate shaft (10).
45. The device (1) according to any one of the preceding aspects, wherein a distal portion (86) of each of the flaps (81) is connected to the elongate shaft (10) at a second position, optionally via a second pushing member collar (87), optionally wherein the second position is distal from the first position, optionally wherein the second pushing member collar (87) is movable with respect to the elongate shaft (10) along the longitudinal axis (A_{L}).
46. The device (1) according to the preceding aspect, wherein the proximal and distal portion (84, 86) of each flap (81) is connected via a hinge portion (88), optionally wherein the flaps (81) bend at the hinge portion (88) when the pushing member (80) is moved to its expanded configuration, optionally wherein the hinge portion (88) has a lower resistance to bending than the proximal and distal portions (84, 86).
47. The device (1) according to any one of the preceding sixteen aspects, wherein the at least one pushing member (80), optionally the at least one flap (81), comprises at least one marker for determining the configuration of the pushing member in a medical imaging procedure, optionally wherein the marker is an ultrasound marker and/or a radiopaque material.
48. The device (1) according to any one of the preceding seventeen aspects, wherein the at least one pushing member (80), optionally the at least one flap (81), is made from an ultrasound visible material and/or a radiopaque material.
49. The device (1) according to any one of the preceding eighteen aspects, wherein the at least one pushing member (80) is provided as or comprises an inflatable structure, for example an inflatable balloon.
50. The device (1) according to the preceding aspect, wherein the flaps (81) are expanded by inflating the inflatable structure, for example with a gas or a liquid.
51. The device (1) according to any one of the preceding aspects, wherein the elongate shaft (10) has a length between 10 mm to 2000 mm, optionally between 50 mm to 500 mm, optionally between 100 mm to 200 mm.
52. The device (1) according to any one of the preceding aspects, wherein the elongate shaft (10) is rigid and/or wherein the elongate shaft (10) is straight along the longitudinal axis (A_{L}).
53. The device (1) according to any one of the preceding aspects, wherein the elongate shaft (10) has a diameter smaller than 15 mm, optionally a diameter smaller than 7 mm, optionally a diameter smaller than 4 mm.
54. The device (1) according to any one of the preceding aspects, wherein the elongate shaft (10) is made from a metallic material or fibre reinforced polymers or by a polymer tube, optionally an extruded polymer tube.
55. The device (1) according to any one of the preceding aspects, wherein the device (1) further comprises a handle at the proximal end (11) of the elongate shaft (10).
56. The device (1) according to any one of the preceding aspects, wherein the device (1) further comprises at least one actuator, optionally a plurality of actuators, at the proximal end (11) of the elongate shaft (10), optionally at the handle.
57. The device (1) according to any one of the preceding aspects, wherein a distal end of the device (1), optionally the distal end (12) of the elongate shaft (10), is atraumatic.
58. The device (1) according to any one of the preceding aspects, wherein a distal portion of the device (1), optionally the distal end (12) of the elongate shaft (10), comprises a plurality of recesses or openings (18), wherein each of the recesses or openings (18) is configured to at least partially receive a respective one of the snares (71, 72) when the snare (71, 72) is advanced out of the respective snare sleeve (61, 62).
59. The device (1) according to any one of the preceding aspects, wherein a distal portion of the device (1), optionally the distal end (12) of the elongate shaft (10), comprises a stop feature (19) for limiting the distal movement of the support structure (20), optionally wherein the stop feature (19) is a protrusion that extends radially outward from the shaft (10).
60. The device (1) according to any one of the preceding aspects, wherein the longitudinal axis (A_{L}) of the elongate shaft (10) is configured to be oriented substantially perpendicular to the tissue (100) when closing the tissue opening (101).
61. The device (1) according to any one of the preceding aspects, wherein the elongate shaft (10) comprises at least three needle guides (13), optionally at least four needle guides (13).
62. The device (1) according to any one of the preceding aspects, wherein the needle guides (13) have a diameter smaller than 2 mm, optionally a diameter smaller than 1 mm.
63. The device (1) according to any one of the preceding aspects, wherein the needle guides (13) have a diameter larger than 0.5 mm, optionally a diameter larger than 0.7 mm.
64. The device (1) according to any one of the preceding aspects, wherein the exit openings (14) are spaced at least 3 mm, optionally at least 8 mm in the proximal direction from a proximally facing side of the support structure (20) that is pressed against the second tissue (100) when the support structure (20) is in the second, expanded configuration.
65. The device (1) according to any one of the preceding aspects, wherein the exit openings (14) are located proximal of a position at which the support structure (20) is coupled to the elongate shaft (10), optionally proximal from a most proximal position at which the support structure (20) is coupled to the elongate shaft (10).
66. The device (1) according to any one of the preceding aspects, wherein each of the needle guides (13) has a first portion (16) and a second portion (17), the second portion (17) extending obliquely to the longitudinal axis (A_{L}) and/or radially outward, optionally wherein the first portion (16) is substantially parallel to the longitudinal axis (A_{L}).
67. The device (1) according to the preceding aspect, wherein the second portion (17) forms a distal portion of the respective needle guide (13).
68. The device (1) according to any of the two preceding aspects,
   wherein each of the needle guides (13) comprises a curved segment, optionally wherein the curved segment is located between the first portion (16) and the second portion (17); and/or
   wherein the second portion (17) is curved.
69. The device (1) according to any one of the preceding aspects,
   wherein each of the needle guides (13) is configured such that the trajectory along which the respective needle (30, 40) extends from the exit opening (14) extends at an angle (α) to the longitudinal axis (A_{L}), optionally:
   wherein the angle (α) is at most 50°, optionally at most 30°; and/or
   wherein the angle (α) is at least 5°, optionally at least 10°.
70. The device (1) according to any one of the preceding aspects, wherein the elongate shaft (10) comprises at least three exit openings (14), optionally at least four exit openings (14), optionally wherein the exit openings (14) are spaced around a periphery of the elongate shaft (10), optionally wherein the exit openings (14) are spaced at regular intervals around the periphery of the elongate shaft (10).
71. The device (1) according to any one of the preceding aspects, wherein the exit openings (14) are located along a peripheral side surface of the elongate shaft (10).
72. The device (1) according to any one of the preceding aspects, wherein the needles (30, 40) are pre-mounted in their respective needle guides (13).
73. The device (1) according to any one of the preceding aspects, wherein the support structure (20) in the first, contracted configuration is configured to be inserted through the tissue opening (101) into the body cavity or lumen (105).
74. The device (1) according to any one of the preceding aspects,
   wherein the support structure (20) in the first, contracted configuration does not radially extend beyond an outer diameter of the elongate shaft (10); and/or
   wherein the support structure (20) in the first, contracted configuration has a diameter of 10 mm or less, optionally 5 mm or less.
75. The device (1) according to any one of the preceding aspects,
   wherein the support structure (20) in the second, expanded configuration extends beyond the outer diameter of the elongate shaft (10); and/or
   wherein the support structure (20) in the second, expanded configuration has a diameter of at least 5 mm, optionally at least 8 mm.
76. The device (1) according to any one of the preceding aspects,
   wherein the support structure (20) is configured to switch between the first, contracted configuration and the second, expanded configuration by pushing and pulling the elongate shaft (10) along the longitudinal axis (A_{L}) and/or by pulling and/or pushing one or more wires extending through or along the elongate shaft (10), respectively; or
   wherein the support structure (20) is configured to switch between the first, contracted configuration and the second, expanded configuration by moving a proximal end of the support structure (20) along the elongate shaft (10) in the proximal direction; and/or
   wherein the support structure (20) is configured to switch between the first, contracted configuration and the second, expanded configuration by pushing and pulling wires extending through the lumen (15) in the elongate shaft (10).
77. The device (1) according to any one of the preceding aspects, wherein the support structure (20) is configured to be expanded by compressing the support structure (20) along the longitudinal axis (A_{L})
78. The device (1) according to any one of the preceding aspects, wherein the support structure (20) comprises a membrane cover and/or one or more inflatable members.
79. The device (1) according to any one of the preceding aspects, wherein the support structure (20) comprises a plurality of support arms (21) such as at least two, at least four, or at least six support arms (21).
80. The device (1) according to the preceding aspect, wherein a proximal portion (22) of each support arm (21) is connected to the elongate shaft (10) at a first position, optionally via a proximal collar (28) of the support structure.
81. The device (1) according to the preceding aspect, wherein the proximal portion (22) of the support arms (21) in the second, expanded configuration is substantially perpendicular to the longitudinal axis (A_{L}).
82. The device (1) according to any of the preceding three aspects, wherein a distal portion (23) of each support arm (21) is connected to the elongated shaft (10) at a second position, optionally via a distal collar (29) of the support structure, optionally wherein the device (1) is configured to be actuated to move the distal portion (23) and/or the distal collar (29) along the elongate shaft (10) in the proximal direction to move the support structure (20) from the first, contracted configuration to the second, expanded configuration.
83. The device (1) according to any of the preceding four aspects, wherein the proximal and distal portions (22, 23) of the support arms (21) are connected via a binge portion (27), optionally wherein the support arms (21) bend at the hinge portion (27) when the support structure (20) is moved to its expanded configuration, optionally wherein the hinge portion (27) has a lower resistance to bending than the proximal and distal portions (22, 23).
84. The device (1) according to any of the preceding five aspects, wherein the proximal and distal portions (22, 23) of the support arms (21) are integrally formed and/or formed as a monolithic component.
85. The device (1) according to any of the preceding six aspects, wherein the support structure (20) is formed as a support basket (24), optionally wherein the support basket (24) is made from nitinol.
86. The device (1) according to the preceding aspect, wherein the support arms (21) are formed by a plurality of struts of the support basket (24), optionally wherein the struts are provided with bending and/or hinge portions (27) having a reduced thickness and/or width.
87. The device (1) according to any of the preceding eight aspects, wherein one or more pairs of the support arms (21) are configured to expand on diametrically opposite sides of the elongate shaft (10).
88. The device (1) according to any of the preceding nine aspects, wherein an extension (61) extends radially outward from each support arm (21) when the support structure (20) is in the second, expanded configuration.
89. The device (1) according to any of the preceding ten aspects, wherein each of the support arms (21) in the first, contracted configuration is substantially parallel to the longitudinal axis (A_{L}).
90. The device (1) according to any of the preceding eleven aspects, wherein, when the first and second needles (30, 40) are in the extended position and the support structure (20) is in the second, expanded configuration, the distal opening of the first needle (30) and the distal opening of the second needle (40) are arranged distally of the first position at which the proximal portion (22) of the support arms (21) is connected to the elongate shaft (10), optionally distal of the proximal collar (28), and proximally of the second position at which the distal portion (23) of the support arms (21) is connected to the elongate shaft (10), optionally proximal of the distal collar (29).
91. The device (1) according to any of the preceding twelve aspects, wherein, when the first and second needles (30, 40) are in the extended position and the support structure (20) is in the second, expanded configuration, the distal opening of the first needle (30) and the distal opening of the second needle (40) are arranged closer to the longitudinal axis (A_{L}) than a radially most-outward portion of the support arms (21).
92. The device (1) according to any of the preceding aspects, wherein, when the first and second needles (30, 40) are in the extended position and the support structure (20) is in the second, expanded configuration, the distal opening of the first needle (30) and the distal opening of the second needle (40) are arranged within an enclosure and/or cage formed by the support structure (20) and/or the support arms (21).
93. The device (1) according to any one of the preceding aspects, wherein the support structure (20), optionally the support arms (21), is/are made from an ultrasound visible material and/or a radiopaque material
94. The device (1) according to any one of the preceding aspects,
   wherein the first needle (30) and the second needle (40) are configured to move separately; or
   wherein the first needle (30) and the second needle (40) are configured to move jointly upon actuation of an actuator of the device (1).
95. The device (1) according to any one of the preceding aspects, wherein the first needle (30) and/or the second needle (40) have a sharp distal end, optionally wherein the distal end has a dull and a sharp portion, optionally wherein the dull portion is provided by a chamfered or radiused edge section.
96. The device (1) according to any one of the preceding aspects, wherein the distal end of the first and second snare sleeves (61, 62) are provided with one or more notches (63).
97. The device (1) according to any one of the preceding aspects, wherein the at least one first needle (30) and/or the at least one second needle (40) comprises a proximal portion and a distal portion, wherein the distal portion is more flexible than the proximal portion.
98. The device (1) according to any one of the preceding aspects, wherein the at least one first needle (30) and/or the at least one second needle (40) comprises a distal portion made from nitinol and/or a proximal portion made from stainless steel, nitinol or titanium.
99. The device (1) according to any one of the preceding aspects, wherein the at least one first needle (30) and/or the at least one second needle (40) comprises a bending portion, optionally wherein a sidewall of the first and/or second needle (30) is provided with a plurality of slots and/or struts along the bending portion, optionally wherein the slots are made by laser cutting.
100. The device (1) according to any one of the preceding aspects, wherein the first needle (30) and/or the second needle (40) has an outer diameter of 0.65 mm to 0.8 mm, and/or an inner diameter of 0.5 mm to 0.65 mm.
101. The device (1) according to any one of the preceding aspects, wherein the device (1) is configured to inject a fluid through the at least one first needle (30) and/or the at least one second needle (40), optionally wherein the fluid contains a growth factor, an anaesthetic, and/or a glue.
102. The device (1) according to any one of the preceding aspects, wherein the device (1) further comprises at least two first needles (30) and at least two second needles (40).
103. The device (1) according to any one of the preceding aspects, wherein the first and/or second snare sleeve (61, 62) is a polymeric tube.
104. The device (1) according to any one of the preceding aspects, wherein the first snare (71) is configured to be closed by advancing the first snare sleeve (61) distally out of the first needle (30) towards the elongate shaft (10) and wherein the second snare (72) is configured to be closed by advancing the second snare sleeve (62) distally out of the second needle (30, 40) towards the elongate shaft (10).
105. The device (1) according to any one of the preceding aspects, wherein a distal end of the snares (71, 72) has an oval shape, an arrow-head shape and/or a tear shape.
106. A method for suturing an opening in a tissue using the device according to any one of the preceding aspects.

Although the present invention has been described with reference to preferred embodiments, workers skilled in the art will recognize that changes may be made in form and detail without departing from the spirit and scope of the invention.

1-15. (canceled)

16. A device for closing a tissue opening in a tissue, the tissue extending at least partially around a body cavity or lumen, the device comprising:
an elongate shaft having a proximal end, a distal end and extending along a longitudinal axis, the elongate shaft configured to be inserted into the tissue opening in the tissue, the elongate shaft comprising at least one first and at least one second needle guide, each needle guide having an exit opening at its distal end;
at least one first hollow needle and at least one second hollow needle, each of the first and second needles being configured to be translated along and advanced out of a respective one of the needle guides in a distal and radially outward direction from a retracted position to an extended position; and
at least one first snare configured to be advanced out of a distal opening of the at least one first hollow needle and at least one second snare configured to be advanced out of a distal opening of the at least one second hollow needle.

17. The device of claim 16, wherein the at least one first snare is configured to be shaped to extend in a radially inward direction towards the elongate shaft when advanced out of a distal opening of the at least one first hollow needle, or wherein the at least one second snare is configured to be shaped to extend in a radially inward direction towards the elongate shaft when advanced out of a distal opening of the at least one second hollow needle.

18. The device of claim 16, wherein the device further comprises:
at least one first snare sleeve configured to be advanced out of a distal opening of the first needle; and
at least one second snare sleeve configured to be advanced out of a distal opening of the second needle.

19. The device of claim 16, wherein the device further comprises
at least one first suture portion and is configured to arrange the first suture portion within the first snare when the first snare is advanced out of the distal opening of the first needle, and
at least one second suture portion and is configured to arrange the second suture portion within the second snare when the second snare is advanced out of the distal opening of the second needle.

20. The device of claim 19, wherein the first and second suture portions are formed as a common suture.

21. The device of claim 19, wherein the first suture portion or the second suture portion is releasably retained proximate a distal end of the shaft in at least one suture holder.

22. The device of claim 21, wherein the first or second suture portion is configured to be pulled out from the suture holder, wherein a length of the first or second suture portion extending into the shaft is configured to be pulled in a proximal direction to release the first or second suture portion from the suture holder.

23. The device of claim 21, wherein the device is configured to allow release of the first suture portion only after the first snare is advanced out of the first needle, or to allow release of the second suture portion only after the second snare is advanced out of the second needle.

24. The device of claim 21, wherein the first or second suture portion is configured to project or bend radially outwards when released from the elongate shaft.

25. The device of claim 19, wherein a segment of the first or second suture portion is stored within a lumen of the shaft.

26. The device of claim 16, wherein the tissue comprises at least first and second tissue layers, the second tissue layer extending between the first tissue layer and the body cavity or lumen, wherein the device further comprises:
a support structure at the distal end of the elongate shaft configured to move between a first, contracted configuration and a second, expanded configuration the support structure being configured to be expanded in the body cavity or lumen, wherein the support structure in the second, expanded configuration is configured to be pressed against the second tissue layer, wherein the first needle is configured to penetrate through the second tissue layer along a first trajectory, the first trajectory extending from the respective exit opening through the second tissue layer obliquely to the longitudinal axis and radially outwards towards the support structure, and the second needle is configured to penetrate through the second tissue layer along a second trajectory, the second trajectory extending from the respective exit opening through the second tissue layer obliquely to the longitudinal axis and radially outwards towards the support structure.

27. The device of claim 26, wherein the device further comprises at least one expandable pushing member configured to push the first tissue layer out of the first and second trajectories, the pushing member being configured to move between a radially contracted configuration and a radially expanded configuration.

28. The device of claim 27, wherein the device is configured to move the pushing member from the contracted configuration to the expanded configuration by compressing the pushing member in the longitudinal direction of the shaft, wherein at least a portion of the first and second trajectories extends through a cavity created between the pushing member and the elongate shaft when the pushing member is in the expanded configuration.

29. The device of claim 27, wherein the at least one pushing member is formed as a pushing member basket.

30. The device of claim 29, wherein the pushing member basket is monolithically formed with a support basket forming the support structure.

31. The device of claim 16, wherein a distal end of the device is atraumatic.

32. The device of claim 18, wherein a distal portion of the device comprises a plurality of recesses or openings, wherein each of the recesses or openings is configured to at least partially receive a respective one of the snares when the snare is advanced out of the respective snare sleeve.

33. The device of claim 16, wherein the device comprises at least two first needles and at least two second needles.

34. The device of claim 18, wherein the first snare is configured to be closed by advancing the first snare sleeve distally out of the first needle towards the elongate shaft and wherein the second snare is configured to be closed by advancing the second snare sleeve distally out of the second needle towards the elongate shaft.

35. A method for suturing an opening in a tissue, the method comprising:
inserting an elongate shaft through the opening into a bodily cavity or lumen;
opening a support structure in the bodily cavity or lumen;
advancing a first hollow needle and a second hollow needle in a distal and radially outward direction from a retracted position to an advanced position;
advancing a first snare out of the first hollow needle, and advancing a second snare out of the second hollow needle;
releasing a first suture portion held at the elongate shaft to move radially outwards into the advanced first snare, and releasing a second suture portion held at the elongate shaft to move radially outwards into the advanced second snare;
closing the first snare with the first suture portion held therein, and closing the second snare with the second suture portion held therein;
retracting the closed first snare through the first hollow needle in a proximal direction with the first suture portion held therein, and retracting the closed second snare through the second hollow needle in a proximal direction with the second suture portion held therein;
retracting the first and second hollow needle and closing the support structure; and
withdrawing the elongate shaft from the opening.
